(19) United States
(12) Patent Application Publication
(10) Pub. No.: US 2005/0136078 A1
(43) Pub. Date: Jun. 23, 2005

(54) Methods for preventing, ameliorating or diminishing buildup of body oil and enhancing its take-up and a cosmetic composition therefor

(76) Inventor: Derrick B. McKie, Brooklyn, NY (US)

Correspondence Address: Charles N.J. Ruggiero, Esq.;Ohlandt, Greeley, Ruggiero & Perle, L.L.P., 10th Floor, One Landmark Square, Stamford, CT, 06901-2682, US

(21) Appl. No.: 10/743,122

(22) Filed: Dec. 22, 2003

(51) Int. Cl. A61K 7/00

(52) U.S. Cl. 424/401

(57) ABSTRACT

There are provided compositions and methods for preventing, ameliorating and/or diminishing oily appearance of skin, lips, and/or hair by topically applying a cosmetic composition having fibers, preferably polymeric fibers, and a cosmetically acceptable vehicle. The fibers are present in an amount effective to take up the body oil. There is also provided methods for enhancing body oil take up of a cosmetic composition. An effective amount of fibers is added to the composition. There is also provided methods for preventing, ameliorating and/or diminishing the oily appearance of skin, lips and/or hair.

### BACKGROUND OF THE INVENTION

I. Field of the Invention

The present invention relates to compositions and methods for preventing, ameliorating, and/or diminishing the buildup of body oil at the surface of the skin, lips, and/or hair. The present invention further relates to methods for enhancing body-oil take-up of a cosmetic composition. The present invention still further relates to methods for preventing, ameliorating, and/or diminishing the oily appearance of the skin, lips, and/or hair.

II. Description of the Related Art

A common problem associated with the care of the skin, lips, and hair is the build-up of body oil on the surface thereof. Although build-up can be diminished with frequent cleanings or washings, the need for such frequent cleanings or washings can be inconvenient. Further, such frequent cleanings or washings can be impossible if a cosmetic composition has recently been topically applied and it is desired that such composition remain on the surface. Cleaning or washing might require that the cosmetic composition be prematurely removed.

U.S. Pat. No. 4,820,510 provides a cosmetic make-up composition having as a binding agent a mixture of 5 wt % to 95 wt % of finely divided silica and about 95 wt % to about 5 wt % of finely divided polyethylene fibers.

U.S. Pat. No. 6,489,283 B1 provides an oil-in-water emulsion having fibers and a surfactant system having at least one glyceryl ester of a C₈ to C₂₄ fatty acid.

U.S. Publication No. 2002/0028222 A1 provides a composition having fibers and a treating active agent for treating greasy skin therein.

It would be desirable to have a composition and a method for preventing, ameliorating or diminishing the buildup of body oil at the surface of the skin, lips, and/or hair. It would also be desirable to have a method for enhancing body-oil take-up of a cosmetic composition. It would be further yet desirable to have a method for preventing, ameliorating, and/or diminishing the buildup of body oil at the surface of the skin, lips, and/or hair.

### SUMMARY OF THE INVENTION

It is an object of the present invention to provide compositions and methods for ameliorating and/or diminishing the buildup of body oil at the surface of the skin, lips, and/or hair.

It is another object of the present invention to provide a composition and a method for preventing the buildup of body oil at the surface of the skin, lips, and/or hair.

It is another object of the present invention to provide a method for enhancing body-oil take-up of a cosmetic composition.

According to these and other objects and advantages of the present invention, there is provided a method for preventing and/or ameliorating and/or diminishing the buildup of body oil at the surface of the skin, lips, and/or hair. A cosmetic composition having a fibrous material and a cosmetically acceptable vehicle is topically applied. The fibrous material is present in an amount effective to take up the body oil.

Further according to these and other objects and advantages of the present invention, there is provided a method for enhancing body-oil take-up of a cosmetic composition. The method includes adding an effective amount of fibrous material to the composition.

Still further according to these and other objects and advantages of the present invention, there is provided a method for preventing and/or ameliorating and/or diminishing the oily appearance of skin, lips and/or hair. The method includes topically applying thereto a cosmetic composition having fibrous material and a cosmetically acceptable vehicle. The fibrous material is present in an amount effective to take up body oil.

Even further according to these and other objects and advantages of the present invention, there is provided a composition for application to skin, lips and/or hair. The composition includes fibrous material in an amount effective to take up body oil and a cosmetically acceptable vehicle.

### DETAILED DESCRIPTION OF THE INVENTION

The present inventor has surprisingly found that there are compositions that ameliorate and/or diminish the buildup of body oil at the surface of the skin, lips, and/or hair. It has also surprisingly been found that there is a composition that prevents the buildup of body oil at the surface of the skin, lips, and/or hair. Accordingly, the present invention provides methods for ameliorating and/or diminishing the buildup of body oil at the surface of the skin, lips, and/or hair, and methods for preventing the buildup of body oil at the surface of the skin, lips, and/or hair. For example, the present invention provides a method for enhancing body-oil take-up of a cosmetic composition, such that the skin, lips and/or hair, look less oily and, thus, less shiny. It further provides a method for ameliorating, and/or diminishing the oily appearance of the skin, lips, and/or hair. The present invention also provides a method for preventing the oily appearance of the skin, lips, and/or hair.

The methods of the present invention employ a fibrous material, preferably polymeric fibers, within cosmetic compositions as “body oil take-up agents” to take up body oil from topical surfaces of the skin, lips, or hair. The terms “take up” and “take-up” are inclusive of absorption, adsorption, or any other mode of capture of the oil by the fibers. The term “body oil” is inclusive of sebum and/or oils secreted by the skin that also can be found on the lips and/or hair follicles. It is also inclusive of mixtures of sebum, oils introduced to the skin, and dirt on the skin, lips, or hair.

The fibrous material is present in the composition at an amount effective to take up body oil at a higher rate and/or to a higher level or degree than compositions not having the fibers. Preferably, the fibrous material is present at about 0.05 weight percent (wt %) to about 30 wt % based on the total weight of the composition. More preferably, the fibrous material is present at about 0.25 wt % to about 15 wt % based on the total weight of the composition. Most preferably, the fibrous material is present at about 0.5 wt % to about 5 wt % based on the total weight of the composition.

The preferred fibers have an average length/diameter ratio of about 1 to about 40. The more preferred fibers have an average length/diameter ratio of about 1 to about 15. Most preferred fibers have an average length/diameter ratio of about 1 to about 5.

The preferred fibers have an average length of about 1 to about 10,000 micrometers. The more preferred fibers have an average length of about 10 to about 1000 micrometers. Most preferred fibers have an average length of about 20 to about 100 micrometers.

The preferred fibers have an average width of about 0.001 to about 100 micrometers. The more preferred fibers have an average width of about 1 to about 50 micrometers. Most preferred fibers have an average width of about 5 to about 30 micrometers.

The fibrous material may be of any fiber that is lipophilic or has an affinity for body oils. The fibers may be of natural or synthetic origin and may be organic or inorganic. The fibers may take the form of individual, discrete fibers or be bundled or agglomerated. Fibers in discrete form are preferred. If the fibers are bundled or agglomerated, they will be bundled or agglomerated in the form of a multiplicity of bundles or agglomerates dispersed throughout the composition. Individual, discrete fibers are likewise dispersed throughout the composition. The fibers may take any cross-sectional shape, such as circular, square, or polygonal. The ends of the fibers may take any configuration, such as blunt, rounded, angled, or shaped. Individual fibers may conform to any geometry lengthwise, such as straight, curly, spiral, and circular.

The fibers that can be used in the present compositions and methods include, but are not limited to the following: silk, cotton, wool, flax, cellulose, polyamide, acetate, polyphenylene terephthalamide, acrylics such as polymethyl methacrylate and poly-2-hydroxyethyl methacrylate, polyolefins such as polyethylene and polypropylene, collagen, polyester, polyvinyl chloride, polyvinylidene chloride, polyvinyl alcohol, polyacrylonitrile, chitosan, polyurethane, or combinations thereof. Fibers of polyolefins, particularly those of polyethylene and polypropylene, are preferred.

In one embodiment of the composition of the present invention, the composition is substantially free of treating active agents, such as those disclosed in paragraph [0043] of U.S. patent Publication 2002/0028222 A1, the paragraph of which is incorporated herein by reference. Treating active agents are not inclusive of fibers, particularly polymeric fibers. Examples of other non-fiber absorptive/adsorptive treating active agents include talc, corn starch, porous silica, silica shells and orgasol, which may function to take up body oil. In yet another embodiment, the composition has less than 0.5 wt % of such non-fiber absorptive/adsorptive treating active agents.

Cosmetic compositions make take any product form suitable for the skin, lips or hair, such as a gel, cream, lotion, liquid, ointment, mask, mousse, pomade, shampoo, solution, spray, stick or solid. Skin compositions can be, but are not limited to, facial cream, body cream, facial mask, cleansing cream, or shaving cream. Hair care compositions may be, but are not limited to, shampoo or conditioner. Lip compositions may be, but are not limited to, cream, balm, and protectant. Make-up compositions may be, but are not limited to, lipstick, lips gloss, rouge, mascara, foundation, blush, and eye shadow.

Cosmetic compositions may be aqueous or anhydrous. The fibers are preferably dispersed or distributed throughout the composition. The composition may take the form of an emulsion or a dispersion. Suitable emulsions include oil-in-water, water-in-oil, water-in-silicone, silicone-in-water, or triple emulsions. Preferred emulsions have about 20 wt % to about 95 wt % water based on the total weight of the composition. More preferred emulsions have about 20 wt % to about 60 wt % water.

Although not essential to the present invention, many methods will require application of the composition for an extended period of time, i.e. about 15 minutes or more, about one hour or more, or about three hours or more, prior to removal, such as by washing, rinsing, wiping or wearing off. The composition will ameliorate and/or diminish build-up of oil on the topical surface, and leave the skin, lips and/or hair looking less oily and, thus, less shiny. It is also logical that the composition will prevent build-up of oil on the topical surface and, again, leave the skin, lips and/or hair looking less oily and, thus, less shiny.

Useful cosmetically acceptable vehicles include, but are not limited to, one or more of the following: water, polyhydric alcohols, monohydric alcohols, silicone oils, mineral oils, vegetable oils, fatty alcohols, fatty acids and esters thereof, or natural and synthetic waxes.

The cosmetic composition may also have one or more antimicrobials, botanical extracts, chelating agents, colorants, conditioners, emollients, emulsifiers, exfoliating agents, fragrances, humectants, insect repellents, lubricants, medicaments, pharmaceutical actives, preservatives, skin whiteners, solubilizing agents, sunscreens, vitamins, waterproofing agents, or any combinations thereof.

The following are examples of the compositions of the present invention:

### EXAMPLE 1

| Part | Ingredients | Wt. % |
|---|---|---|
| A | Glyceryl stearate | 10 |
| | Propylene glycol | 8 |
| | dicaprylate/dicaprate | |
| | Cetearyl alcohol and sodium cetearyl | 5 |
| | sulfate | |
| B | Propylene glycol | 3 |
| | Allantoin | 0.2 |
| | Methylparaben | 0.1 |
| | Polyethylene fibers ESS50 | 5 |
| | Demineralized water | 68.4 |
| C | Fragrance | 0.3 |

The part A components are melted and paddle mixed together at 75° to 80° C. The part B components are separately paddle-mixed and brought to the same temperature as part A. Part A is milled into Part B. The resultant mixture is cooled to 35° C., then the fragrance (Part C) is paddle-mixed therein.

### EXAMPLE 2

| Part | Ingredients | Wt. % |
|---|---|---|
| A | Propylene glycol | 4 |
| | Xanthan Gum | 0.5 |
| | Phenoxyethanol | 0.3 |
| | Demineralized water | 58 |
| | Polypropylene fibers Y600 | 3 |
| B | Squalane | 10 |
| | PPG-12/SMDI | 8 |
| | Hydrogenated phospholipsids | 5 |
| | Caprylic/capric/stearic triglyceride | 2 |
| | Cyclopentasiloxane | 4 |
| | Dimethicone | 1 |
| | Cetearyl alcohol and ceteareth-20 | 2 |
| | Glyceryl stearate and PEG-100 | 1.5 |
| | stearate | |
| | Steareth-2 | 0.5 |
| C | Fragrance | 0.2 |

For part A, the xanthan gum is slowly dispersed in the water while vigorously stirring. Mixing is continued until the gum is thoroughly dissolved. The polypropylene fibers are then paddle-dispersed into the water phase. The mixture is heated 75° C., then the propylene glycol is added to it followed by the phenoxyethanol.

The components of part B are combined in a separate vessel and slowly mixed while heating to 75° C. Part B is slowly milled into part A, then the batch is cooled to 35° C. The fragrance is then paddle-mixed into the batch.

Having thus described the present invention with particular referenced to preferred embodiments thereof, it will be apparent that various changes and modifications may be made therein without departing from the spirit and scope of the present invention as defined in the appended claims.

1. A method for ameliorating and/or diminishing the buildup of body oil at the surface of the skin, lips, and/or hair, comprising:
topically applying a cosmetic composition having fibers that act as a body oil take-up agent and a cosmetically acceptable vehicle, wherein the fibers are present in an amount effective to take up the body oil.

2. The method of claim 1, wherein the fibers are present at about 0.05 wt % to about 30 wt % based on the total weight of the composition.

3. The method of claim 1, wherein the fibers are present at about 0.25 wt % to about 15 wt % based on the total weight of the composition.

4. The method of claim 1, wherein the fibers have an average length/diameter ratio of about 1 to about 40.

5. The method of claim 1, wherein the fibers have an average length of about 10 to about 1000 micrometers, and an average width of about 1 to about 50 micrometers.

6. The method of claim 1, wherein the fibers are polyolefin fibers.

7. The method of claim 1, wherein the fibers are selected from the group consisting of polyethylene fibers, polypropylene fibers, and combinations thereof.

8. The method of claim 1, wherein the fibers are selected from the group consisting of silk, cotton, wool, flax, cellulose, polyamide, acetate, polyphenylene terephthalamide, acrylics, polymethyl methacrylate, poly-2-hydroxyethyl methacrylate, polyolefin, polyethylene, polypropylene, collagen, polyester, polyvinyl chloride, polyvinylidene chloride, polyvinyl alcohol, polyacrylonitrile, chitosan, polyurethane, and any combinations thereof.

9. The method of claim 1, wherein the composition is an emulsion.

10. The method of claim 1, wherein the composition is a dispersion.

11. The method of claim 1, wherein the composition is in a product form selected from the group consisting of a gel, a cream, a lotion, a liquid, a mousse, a solution, a spray, an ointment, a mask, a shampoo, a pomade, a stick, and a solid.

12. The method of claim 1, wherein the cosmetic composition is a make-up composition selected from the group consisting of a lipstick, a lips gloss, a rouge, a mascara, a foundation, a blush, and an eye shadow.

13. The method of claim 1, wherein the cosmetic composition is a skin composition selected from the group consisting of a facial cream, a body cream, a facial mask, a cleansing cream, and a shaving cream.

14. The method of claim 1, wherein the composition is permitted to remain on the surface of the skin, lips, and/or hair for about 15 minutes or more.

15. The method of claim 1, wherein the composition is permitted to remain on the surface of the skin, lips, and/or hair for about one hour or more.

16. The method of claim 1, wherein the composition is permitted to remain on the surface of the skin, lips, and/or hair for about three hours or more.

17. A method for enhancing body-oil take-up of a cosmetic composition, comprising adding to the composition an effective amount of fibers that act as a body oil take-up agent.

18. A method for ameliorating and/or diminishing the oily appearance of skin, lips and/or hair, comprising:
topically applying thereto a cosmetic composition having fibers that act as a body oil take-up agent and a cosmetically acceptable vehicle, wherein the fibers are present in an amount effective to take up body oil.

19. A composition for application to skin, lips and/or hair, comprising:
a sebum absorption/absorption treating agent consisting of fibers that take up body oil, wherein the fibers are present in an amount effective to take up body oil, and
a cosmetically acceptable vehicle.

20. The composition of claim 19, wherein the fibers have an average length/diameter ratio of about 1 to about 40.

21. The composition of claim 19, wherein the fibers have an average length of about 10 to about 1000 micrometers, and an average width of about 1 to about 50 micrometers.

22. The composition of claim 19, wherein the composition is an emulsion.

23. A method for ameliorating or diminishing the buildup of body oil at the surface of the skin, lips, and/or hair, comprising:
topically applying a cosmetic composition having a body oil take-up agent and a cosmetically acceptable vehicle, wherein the body oil take-up agent consists essentially of fibers, and wherein the body oil take-up agent is present in an amount effective to take up the body oil.

24. A method for enhancing body-oil take-up of a cosmetic composition, comprising adding to the composition an effective amount of a body oil take-up agent consisting essentially of fibers.

25. A method for ameliorating or diminishing the oily appearance of skin, lips and/or hair, comprising:
topically applying thereto a cosmetic composition having a body oil take-up agent and a cosmetically acceptable vehicle, wherein the body oil take-up agent consists essentially of fibers, and wherein the body oil take-up agent is present in an amount effective to take up body oil.

26. A method for preventing the buildup of body oil at the surface of the skin, lips, and/or hair, comprising:
topically applying a cosmetic composition having fibers that act as a body oil take-up agent and a cosmetically acceptable vehicle, wherein the fibers are present in an amount effective to take up the body oil.

27. The method of claim 1, wherein the composition is substantially free of a non-fiber sebum absorptive/adsorptive sebum active agent.
